(19) United States
(12) Plant Patent Application Publication
(10) Pub. No.: US 2009/0235415 P1
(43) Pub. Date: Sep. 17, 2009

(54) Raspberry Plant Named 'Erika'

(50) ;

(76) Inventors: Antonio Pititto, Civezzano (IT); Luigi Gadler, Pergine Valsugana (IT); Flavio Roberto De Salvador, Marino (IT)

Correspondence Address: JONDLE & ASSOCIATES P.C., 858 HAPPY CANYON ROAD SUITE 230, CASTLE ROCK, CO, 80108, US

(21) Appl. No.: 12/075,346

(22) Filed: Mar. 11, 2008

(51) Int. Cl. A01H 5/00

(52) U.S. Cl. PLT/204

(57) ABSTRACT

A new Raspberry plant particularly distinguished by being a primocane raspberry plant having favourable fruit color and flavour, large and firm berry, and good tolerance to raspberry pests and diseases, is disclosed.

### GENUS AND SPECIES

Rubus idaeus L.

### VARIETY DENOMINATION

‘Erika’

### BACKGROUND OF THE NEW PLANT

The present invention comprises a new and distinct cultivar of red raspberry, botanically known as Rubus idaeus L., and hereinafter referred to by the cultivar name ‘Erika’. ‘Erika’ was produced from the cross of the female parent ‘Autumn Bliss’ (U.S. Plant Pa. 6,597) with an unknown individual raspberry plant.

The crossing for the new cultivar occurred in 2002, in Trentino, Italy and has been asexually reproduced repeatedly by root cuttings since May 2007. The present invention has been found to retain its distinctive characteristics through successive asexual propagations in Trentino, Italy.

Plant breeder's rights were applied for with the European Union on Mar. 12, 2007. ‘Erika’ has not been made publicly available more than one year prior to filing of this application.

### SUMMARY OF THE INVENTION

The following are the most outstanding and distinguishing characteristics of this new cultivar when grown under normal horticultural practices in Trentino, Italy.
a. Primocane raspberry plant;
b. Favorable fruit color and flavor;
c. Large and firm berry; and
d. Good tolerance to raspberry pests and diseases.

### DESCRIPTION OF THE PHOTOGRAPHS

This new raspberry plant is illustrated by the accompanying photographs which show the fruit of the plant as well as the primocanes; the colors shown are as true as can be reasonably obtained by conventional photographic procedures. The photographs are of plants that are 2 years old.

FIG. 1 shows sections of the canes.

FIG. 2 shows the mature fruit.

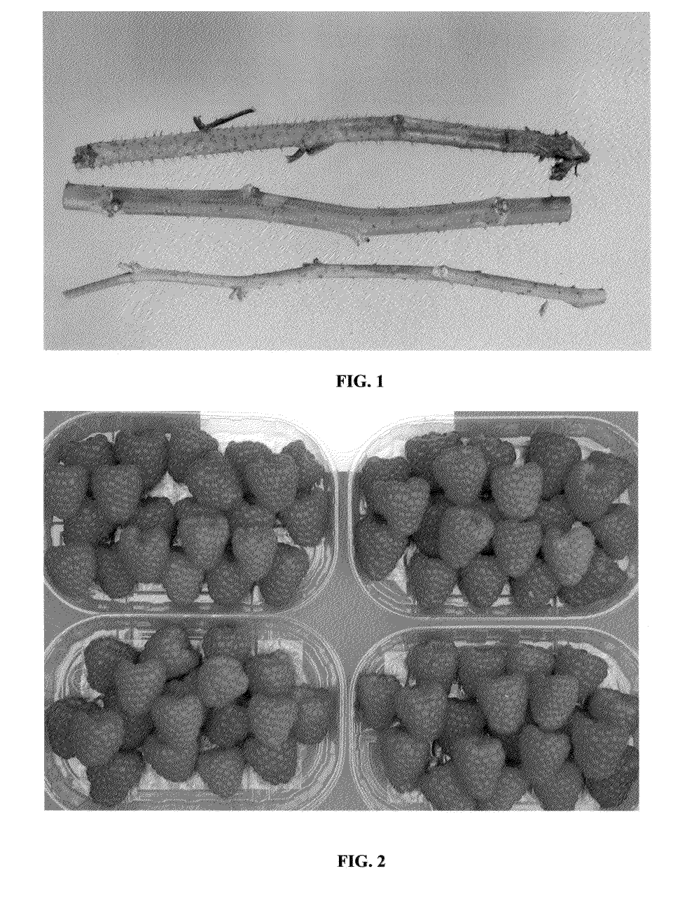

### DESCRIPTION OF THE NEW CULTIVAR

The following detailed descriptions set forth the distinctive characteristics of ‘Erika’. The data which define these characteristics were collected from root cuttings in Trentino, Italy. The plant history was taken on plants grown for 2 years under field conditions with natural light during the Autumn or primocane season.

### DETAILED BOTANICAL DESCRIPTION

Classification:
   Family.—Rosaceae.
   Botanical.—Rubus idaeus L.
   Common name.—Raspberry.
Parentage:
   Female parent.—Raspberry plant ‘Autumn Bliss’ (U.S. Plant Pat. No. 6,597).
   Male parent.—Unknown individual raspberry plant.
Plant:
   Propagated.—Root cuttings Self-fertile.
Primocanes:
   Cane color.—Brown.
   Spines.—Present, thin and dense.
Fruit:
   Predominant shape.—Broad-conical.
   Weight.—4.85 g.
   Size.—Length: 2.341 cm Width: 2.176 cm.
   Color of mature fruit.—Light-red.
   Firmness (Newtons/mm).—2.18.
   Vigor.—Medium.
   Harvest time.—Meduim-early.
   Time of ripening.—8 days earlier than ‘Polka’.
   Fruit grading % (percentage of fruit greater than 22 mm).—10 to 20%.
   Production (kg/plant).—20 to 30% higher yield than ‘Polka’.
   Tritratable acidity (meq/l).—25.58.
   Brix.—6.27.
Disease and insect resistance: Good tolerance has been observed to typical raspberry pests and diseases.
Comparison with parental and commercial cultivars: ‘Erika’ differs from the parental variety ‘Autumn Bliss’ (U.S. Plant Pat. No. 6,597) in that ‘Erika’ has a broad-conical shaped, light-red colored fruit weighing 4.85 g, while ‘Autumn Bliss’ has a oval-conical shaped, medium to dark-red colored fruit weighing between 3.5 g to 4.0 g. ‘Erika’ is similar to the commercial variety ‘Polka’ (unpatented), however, as shown in Table 1, there are numerous differences.

TABLE 1
Characteristic Comparison between ‘Erika’ and ‘Polka’

| Characterisstic | ‘Erika’ | ‘Polka’ |
|---|---|---|
| Plant vigor | Medium | Medium-high |
| Canes color | Brown | Light-brown |
| Spines | Thin and dense | Thick and short |
| Fruit shape | Broad-conical | Conical |
| Ripening time | 8 days earlier than ‘Polka’ | Early (day 0) |
| Fruit color | Light-red | Red |

I claim:

1. A new and distinct cultivar of Raspberry plant as shown and described herein.
